(19) United States
(12) Patent Application Publication
(10) Pub. No.: US 2025/0235910 A1
(43) Pub. Date: Jul. 24, 2025

(54) AN IMPROVED DISINFECTION-SOLIDIFICATION PROCESS FOR PATHOGENIC MEDICAL WASTE DISPOSAL

(71) Applicant: Council of Scientific and Industrial Research, New Delhi (IN)

(72) Inventors: Sreejith Shankar Pooppanal, Thiruvananthapuram (IN); Sruthi Surendran Nair, Thiruvananthapuram (IN); Suja Pottath, Thiruvananthapuram (IN); Hareesh Unnikrishnan Nair Saraswathy, Thiruvananthapuram (IN); Rajeev Kumar Sukumaran, Thiruvananthapuram (IN); Savithri Sivaraman, Thiruvananthapuram (IN); Parukkuttyamma Devi Sujatha, Thiruvananthapuram (IN); Ajayaghosh Ayyappanpillai, Thiruvananthapuram (IN)

(73) Assignee: Council of Scientific and Industrial Research, New Delhi (IN)

(21) Appl. No.: 18/698,530

(22) PCT Filed: Aug. 17, 2022
(86) PCT No.: PCT/IN2022/050745

§ 371 Date: Apr. 4, 2024

(30) Foreign Application Priority Data
Oct. 4, 2021 (IN) 202111045340

(51) Int. Cl. B09B 3/10; B09B 3/40; B09B 101/65

(52) U.S. Cl. CPC B09B 3/10; B09B 3/40; B09B 2101/65

(57) ABSTRACT

The present invention discloses an improved process for the efficient solidification of biomedical waste that is capable of simultaneously treating and disinfecting solid and fluid samples. The process comprises of the addition of the waste samples to an alkaline aqueous solution followed by the addition of a solid material at a defined volumetric and/or weighted composition leading to instantaneous solidification with >99.9% microbial disinfection and an all-in-one disinfecting device 10 for treatment of biomedical waste.

### FIELD OF THE INVENTION

The present invention relates to an improved process for the efficient solidification of biomedical waste that is capable of simultaneously treating and disinfecting solid and fluid samples. Particularly, the present invention relates to the process for disinfecting biomedical wastes comprising of the addition of the waste samples to an alkaline aqueous solution followed by the addition of a solid material at a defined volumetric and/or weighted composition leading to instantaneous solidification with >99.9% microbial disinfection. More particularly, the present invention relates to a disinfecting device for treatment of biomedical waste.

### BACKGROUND AND PRIOR ART OF THE INVENTION

Mismanagement of infectious wastes such as biomedical test samples leads to the transmission of microbes/toxins/viruses and further steer the spread of contagious and infectious diseases. According to a position statement (2000) by WHO, improper management of medical wastes such as infected hypodermic needles and syringes has caused infections pertaining to hepatitis B (21 million cases), hepatitis C (2 million cases) and HIV (0.26 million cases) worldwide. The following statements quoted from WHO undermine the significance and need for proper medical waste management: “Poor management of medical waste potentially exposes healthcare workers, waste handlers, patients and the community at large to infection, toxic effects and injuries, and risks polluting the environment. It is essential that all medical waste materials are segregated at the point of generation, appropriately treated, and disposed of safely” (Reproduced from http://www.who.int/topics/medical_waste/en/).

Adding a flocculating agent to liquid waste reduces the risk of spills and aerosolization. Solid wastes such as cotton, sharps as well as tissue papers may also lead to spread of infections and simple absorbers or hypochlorites that are currently in use are not always capable of treating such wastes. If the flocculating/gelling agent contains a disinfectant, it may be possible to dispose of the waste as non-regulated medical waste, which is less expensive than red-bagging. Segregation, transportation and incineration of such disinfected medical wastes are easier, safer and decrease medical waste disposal costs for a healthcare facility.

Several strategies have been adopted for the management of liquid biomedical waste and include, but not limited to, sanitary sewer disposal methods, chemical treatment using 1% sodium hypochlorite solution with a minimum contact period of 30 min or 10-14 g bleaching powder per liter of water, 70% ethanol, 4% formaldehyde, 70% isopropyl alcohol, 25% iodine or 6% hydrogen peroxide, solidification of liquid waste using dry super adsorbent polymers containing sanitizers or disinfecting agents like chlorine or glutaraldehyde, closed disposal systems, etc. Reference may be made to an article, “Liquid biomedical waste management: An emerging concern for physicians, Biswal S, Muller J Med Sci Res2013, 4, 99-106 which states that the culture media containing high microbial loads or rich protein contents requires rigorous disinfection procedures, wherein inactivation is achieved using 5.23% sodium hypochlorite in a 1:10 dilution for a minimum of 8 h inside a secured vessel followed by disposal down the sanitary sewer and subsequent flushing with a lot of cold water for at least of 10 min.(., ,

Solidification systems (super adsorbents) are deemed advantageous over other methods for the treatment and safer disposal of biomedical fluid wastes. Superabsorbent polymers are generally prepared polymerizing unsaturated carboxylic acids or derivatives thereof, including, but not limited to, acrylic acid or its or metal/ammonium salts and alkyl acrylates, using an internal cross-linking agent such as oligo-functional monomers including, but not limited to, bisacrylamides, triacrylates, dimethacrylates, or triallylamines.

Several patents have educated the development of such solidification systems. Application Reference may bemade to the U.S. Pat. No. 7,291,674B2 wherein surface cross-linked superabsorbent polymers with good liquid retention, permeability, and mechanical strength based on the absorbent structure.

Reference may be made to the U.S. Pat. No. 8,450,389B1, wherein one or a plurality of surface cross-linked superabsorbent particles in combination with a plurality of second particles for liquid solidification with reduced gel block and a method of solidifying liquid medical waste Reference may be made to the another patent U.S. Pat. No. 9,533,081B1, wherein a portable wound therapy system comprising a plurality of surface cross-linked superabsorbent particles along with a container, a wound covering, and a packet and included a similar liquid solidification system with reduced gel block.

Reference may be made to thepatent U.S. Pat. No. 5,391,351A, wherein a body waste fluid solidification device comprising a hydrophilic xerogel of partially hydrolyzedpoly(vinyl acetate), cross-linked poly(vinyl alcohol), cross-linked hydroxyalkyl acrylates and methacrylates, polymers and copolymers of ethylene oxide and polymers and copolymers acrylamide

Reference may be made to the U.S. Pat. No. 6,797,857B2, wherein a solidifier for the solidification of a volume of liquid with a known density, comprising of three adsorbents with varying densities, thereby achieving controlled stabilization of a flowable material throughout its overall volume.

Reference may be made to the U.S. Pat. No. 5,424,265A, wherein a capsule for absorbing liquid waste with a powder adsorbent material disposed within said capsule, the body of the said capsule being water soluble leads to the adsorption of liquid waste located within a suction canister.

Reference may be made to the U.S. Pat. No. 9,102,806B2, wherein a particulate superabsorbent polymer capable of absorbing water, aqueous liquids, and blood, and a process to manufacture the said superabsorbent polymers. The said super adsorbent comprises of a 1-10 wt % of a thermoplastic polymer of any class selected from polyolefin, polyethylene, linear low density polyethylene, ethylene acrylic acid copolymer, styrene copolymers, ethylene alkyl methacrylate copolymer, polypropylene, ethylene vinyl acetate copolymer, polyamide, polyester, blends thereof, or copolymers thereof, where the surface is treated with a neutralized multivalent metal salt solution having a pH value similar to that of human skin.

Reference may be made to the U.S. Pat. No. 8,403,904B2, wherein a superabsorbent polymer comprising an internal cross-linking agent consisting of a silane derivative having a minimum of one vinyl group or one allyl group attached to a silicon atom, and at least one Si—O bond with high centrifuge retention capacity.

Super adsorbing polymers, methods for their preparation and application in liquid solidification have been described by several patents, viz, EP2739660B2, US20130310251A1, EP0273141B1,U.S. Pat. No. 8,476,189B1, JP5527916B2, U.S. Pat. No. 5,578,318A, DE69815670T2, U.S. Pat. No. 8,821,363B1.

Solid wastes including, but not limited to, used cotton, tissue papers, syringes and needles are generally disinfected using approved disinfectants and/or sanitizers and are incinerated or recycled.

Waste burial or land-fills, disposal in cemented pits, immobilization using plastic foam, sand, cement or clay, low/medium/high temperature burning, controlled incineration, steam autoclaving, rotary kiln, microwave treatment, chemical treatment, shredding, melting, etc. are the general practices in disposing solid waste (Reference may be made to WHO @www.who.int/, and Medical Waste Management, International Committee of the Red Cross @www.icrc.org/). A 1-10% solution of bleach, or hypochlorites, sodium hydroxide or other chemical disinfectants are used to disinfect biomedical waste. Heat, alkaline digesters and microwaves are also used for this purpose.

Acrylate based solidifiers, though cheap and vastly available, are not devoid of disadvantages. It generally takes 10-15 min. for complete gelation and are not easily recycled. They are non-biodegradable and some acrylates are shown to be flammable. Studies have indicated that several acrylates and their raw materials can be carcinogenic. Manufacturing of acrylics has both health and environmental impacts. Several chemicals used in the manufacturing as well as the chemical waste from acrylic plants are toxic. Hypochlorite (bleach) is not always effective with high organic content waste such as blood. Further, a disinfection system capable of instantaneously treating, immobilizing and disinfecting both liquid and solid medical wastes is not found in literature.

### ABBREVIATIONS USED

WHO: World Health Organization
min. : minutes
wt %: weight percentage
NaOH: Sodium hydroxide
mg: milligram
mL: milliliter
kg: kilogram

### OBJECTIVES OF THE INVENTION

The primary objective of the present invention relates to the development of an efficient solidification system that is capable of simultaneously treating and disinfecting solid and fluid samples.

Another objective is to provide a process for the preparation for disposal of solid and fluid waste collected in a container or a collection vessel at the required point of care.

A third objective is to provide an easy, safe and cost-effective strategy for reducing the risks of spillage and occupational exposure thereby providing a process for managing biomedical wastes, including both solid and liquid wastes.

Yet another objective is to develop a process for the preparation for disposal of solid and fluid waste by destroying or disinfecting or deactivating the infectious agents in the wastes for the preparation for disposal including treatment and transport of the samples after solidification.

### SUMMARY OF THE INVENTION

In view of the above technical background, the present invention intends to disclose an improved process for the disinfection and solidification of biomedical waste. The process involves the use of solid powders of a solidifying agent and a basifying solution, which when subjected to mixing with solid or fluid waste samples at a defined volumetric and/or weighted composition leads to instantaneous solidification with up to 100% microbial disinfection.

The present invention intends to provide a disinfection system for the preparation for disposal of solid and fluid wastes collected in a collection vessel combined with the destruction, disinfection or deactivation of infectious agents including microorganisms inter alia bacteria, fungus etc., viruses and other toxins, whereby the disposal including treatment, handling and transportation are deemed easier, safer and cost-effective.

Another object of the present invention provides a method to create a non-pourable environment for fluid medical wastes inter alia salt, sugar, saliva, urine, blood, hospital chemicals, etc. wherein risks related to spillage and occupational exposure are minimized, and further to the treatment of solid medical wastes inter alia cotton, tissue paper, swabs, needles, etc., wherein the risks related to accumulation of untreated and infected samples are minimized or a mixture of solid and liquid wastes added with >99.9% microbial disinfection.

In another embodiment, the present invention discolses the process involving an aqueous solution of a pH regulating base or alkali for complete disinfection of fluid or solid medical waste followed by the addition of an oxide based solid powder, as a single or plurality of the said powders, for instantaneous solidification of solid or fluid samples containing proteins, microbial cultures, salt or metal ions in high concentrations.

In a final object, the invention intends to create all-in-one sample collection—disinfection—solidification devices of requisite dimensions capable of collecting the solid or liquid sample, and immobilizing them as and when required with prior pathogenic disinfection for preparation for its disposal.

### BRIEF DESCRIPTION OF THE FIGURES

FIG. 1 illustrates the solidification process involving saturated salt (NaCl) solution upon addition of silica gel (chromatographic grade, 60-120 mesh): (a) 1 mL 50% aqueous NaOH, (b) 1 mL saturated salt solution, (c) 1 mL 50% aqueous NaOH+1 mL saturated salt solution and (d) after addition of silica gel for solidification.

FIG. 2 illustrates the solidification process involving saturated sugar (sucrose) solution upon addition of silica gel (chromatographic grade, 60-120 mesh): (a) 1 mL 50% aqueous NaOH, (b) 1 mL saturated sugar solution, (c) 1 mL 50% aqueous NaOH+1 mL saturated sugar solution and (d) after addition of silica gel for solidification.

FIG. 3 illustrates the solidification process involving a mixture of saturated salt (NaCl) and sugar (sucrose) solutions upon addition of silica gel (chromatographic grade, 60-120 mesh): (a) 1 mL 50% aqueous NaOH, (b) 0.5 mL saturated salt+0.5 mL saturated sugar solutions, (c) 1 mL 50% aqueous NaOH+1 mL saturated salt+sugar solutions and (d) after addition of silica gel for solidification.

FIG. 4 illustrates the solidification process involving 6% BSA solution upon addition of silica gel (chromatographic grade, 60-120 mesh): (a) 1 mL 50% aqueous NaOH, (b) 6% BSA solution, (c) 1 mL 50% aqueous NaOH+1 mL 6% BSA solution and (d) after addition of silica gel for solidification.

FIG. 5 illustrates the solidification process involving a mixture of saturated salt (NaCl) and 6% BSA solutions upon addition of silica gel (chromatographic grade, 60-120 mesh): (a) 1 mL 50% aqueous NaOH, (b) 0.5 mL saturated salt+0.5 mL 6% BSA solutions, (c) 1 mL 50% aqueous NaOH+1 mL saturated salt+6% BSA solutions and (d) after addition of silica gel for solidification.

FIG. 6 illustrates the solidification process involving saturated potassium dichromate solution upon addition of silica gel (chromatographic grade, 60-120 mesh): (a) 1 mL 50% aqueous NaOH, (b) 1 mL saturated potassium dichromate solution, (c) 1 mL 50% aqueous NaOH+1 mL saturated potassium dichromate solution and (d) after addition of silica gel for solidification.

FIG. 7 illustrates the solidification process involving iodine solution upon addition of silica gel (chromatographic grade, 60-120 mesh): (a) 1 mL 50% aqueous NaOH, (b) 1 mL iodine solution, (c) 1 mL 50% aqueous NaOH+1 mL iodine solution and (d) after addition of silica gel for solidification.

FIG. 8 illustrates the solidification process involving artificial blood upon addition of silica gel (chromatographic grade, 60-120 mesh): (a) 1 mL 50% aqueous NaOH, (b) 1 mL artificial blood, (c) 1 mL 50% aqueous NaOH+1 mL artificial blood and (d) after addition of silica gel for solidification. 6% BSA provided the protein content and heme was substituted with a iron(II) complex.

FIG. 9 illustrates the solidification process involving artificial urine upon addition of silica gel (chromatographic grade, 100-200 mesh): (a) 1 mL 50% aqueous NaOH, (b) 1 mL 50% aqueous NaOH+1 mL artificial urine and (c) after addition of silica gel for solidification.

FIG. 10 illustrates the solidification process involving artificial saliva upon addition of silica gel (chromatographic grade, 100-200 mesh): (a) 1 mL 50% aqueous NaOH, (b) 1 mL 50% aqueous NaOH+1 mL artificial saliva and (c) after addition of silica gel for solidification.

FIG. 11 illustrates the solidification process involving saturated salt (NaCl) solution upon addition of silica gel (chromatographic grade, 100-200 mesh): (a) 1 mL 50% aqueous NaOH, (b) 1 mL saturated salt solution, (c) 1 mL 50% aqueous NaOH+1 mL saturated salt solution and (d) after addition of silica gel for solidification.

FIG. 12 illustrates the solidification process involving saturated sugar (sucrose) solution upon addition of silica gel (chromatographic grade, 100-200 mesh): (a) 1 mL 50% aqueous NaOH, (b) 1 mL saturated sugar solution, (c) 1 mL 50% aqueous NaOH+1 mL saturated sugar solution and (d) after addition of silica gel for solidification.

FIG. 13 illustrates the solidification process involving 6% BSA solution upon addition of silica gel (chromatographic grade, 100-200 mesh): (a) 1 mL 50% aqueous NaOH, (b) 6% BSA solution, (c) 1 mL 50% aqueous NaOH+1 mL 6% BSA solution and (d) after addition of silica gel for solidification.

FIG. 14 illustrates the solidification process involving saturated potassium dichromate solution upon addition of silica gel (chromatographic grade, 100-200 mesh): (a) 1 mL 50% aqueous NaOH, (b) 1 mL saturated potassium dichromate solution, (c) 1 mL 50% aqueous NaOH+1 mL saturated potassium dichromate solution and (d) after addition of silica gel for solidification.

FIG. 15 illustrates the solidification process involving iodine solution upon addition of silica gel (chromatographic grade, 100-200 mesh): (a) 1 mL 50% aqueous NaOH, (b) 1 mL iodine solution, (c) 1 mL 50% aqueous NaOH+1 mL iodine solution and (d) after addition of silica gel for solidification.

FIG. 16 illustrates the solidification process involving artificial blood upon addition of silica gel (chromatographic grade, 230-400 mesh): (a) 1 mL 50% aqueous NaOH, (b) 1 mL artificial blood, (c) 1 mL 50% aqueous NaOH+1 mL artificial blood and (d) after addition of silica gel for solidification. 6% BSA provided the protein content and heme was substituted with a iron(II) complex.

FIG. 17 illustrates the solidification process involving artificial urine upon addition of silica gel (chromatographic grade, 100-200 mesh): (a) 1 mL 50% aqueous NaOH, (b) 1 mL 50% aqueous NaOH+1 mL artificial urine and (c) after addition of silica gel for solidification.

FIG. 18 illustrates the solidification process involving artificial saliva upon addition of silica gel (chromatographic grade, 100-200 mesh): (a) 1 mL 50% aqueous NaOH, (b) 1 mL 50% aqueous NaOH+1 mL artificial saliva and (c) after addition of silica gel for solidification.

FIG. 19 illustrates the solidification process involving saturated salt (NaCl) solution upon addition of silica gel (chromatographic grade, 230-400 mesh): (a) 1 mL 50% aqueous NaOH, (b) 1 mL saturated salt solution, (c) 1 mL 50% aqueous NaOH+1 mL saturated salt solution and (d) after addition of silica gel for solidification.

FIG. 20 illustrates the solidification process involving saturated sugar (sucrose) solution upon addition of silica gel (chromatographic grade, 230-400 mesh): (a) 1 mL 50% aqueous NaOH, (b) 1 mL saturated sugar solution, (c) 1 mL 50% aqueous NaOH+1 mL saturated sugar solution and (d) after addition of silica gel for solidification.

FIG. 21 illustrates the solidification process involving 6% BSA solution upon addition of silica gel (chromatographic grade, 230-400 mesh): (a) 1 mL 50% aqueous NaOH, (b) 6% BSA solution, (c) 1 mL 50% aqueous NaOH+1 mL 6% BSA solution and (d) after addition of silica gel for solidification.

FIG. 22 illustrates the solidification process involving saturated potassium dichromate solution upon addition of silica gel (chromatographic grade, 230-400 mesh): (a) 1 mL 50% aqueous NaOH, (b) 1 mL saturated potassium dichromate solution, (c) 1 mL 50% aqueous NaOH+1 mL saturated potassium dichromate solution and (d) after addition of silica gel for solidification.

FIG. 23 illustrates the solidification process involving iodine solution upon addition of silica gel (chromatographic grade, 230-400 mesh): (a) 1 mL 50% aqueous NaOH, (b) 1 mL iodine solution, (c) 1 mL 50% aqueous NaOH+1 mL iodine solution and (d) after addition of silica gel for solidification.

FIG. 24 illustrates the solidification process involving artificial blood upon addition of silica gel (chromatographic grade, 230-400 mesh): (a) 1 mL 50% aqueous NaOH, (b) 1 mL artificial blood, (c) 1 mL 50% aqueous NaOH+1 mL artificial blood and (d) after addition of silica gel for solidification. 6% BSA provided the protein content and heme was substituted with a iron(II) complex.

FIG. 25 illustrates the solidification process involving artificial urine upon addition of silica gel (chromatographic grade, 230-400 mesh): (a) 1 mL 50% aqueous NaOH, (b) 1 mL 50% aqueous NaOH+1 mL artificial urine and (c) after addition of silica gel for solidification.

FIG. 26 illustrates the solidification process involving artificial saliva upon addition of silica gel (chromatographic grade, 230-400 mesh): (a) 1 mL 50% aqueous NaOH, (b) 1 mL 50% aqueous NaOH+1 mL artificial saliva and (c) after addition of silica gel for solidification.

FIG. 27 illustrates the solidification process involving saturated potassium dichromate solution upon addition of alumina (chromatographic grade, basic, 60-325 mesh): (a) 1 mL 50% aqueous NaOH, (b) 1 mL saturated potassium dichromate solution, (c) 1 mL 50% aqueous NaOH+1 mL saturated potassium dichromate solution and (d) after addition of alumina for solidification.

FIG. 28 illustrates the solidification process involving saturated potassium dichromate solution upon addition of titania (mixture of anatase and rutile): (a) 1 mL 50% aqueous NaOH, (b) 1 mL saturated potassium dichromate solution, (c) 1 mL 50% aqueous NaOH+1 mL saturated potassium dichromate solution and (d) after addition of titania for solidification.

FIG. 29 illustrates the solidification process involving cotton pieces upon addition of silica gel (chromatographic grade): (a) 1 mL 50% aqueous NaOH+a piece of cotton, and after addition of silica gel for solidification: (b) 60-120 mesh, (c) 100-200 mesh and (d) 230-300 mesh.

FIG. 30 illustrates the solidification process involving tissue paper upon addition of silica gel (chromatographic grade): (a) 1 mL 50% aqueous NaOH+a piece of tissue paper, and after addition of silica gel for solidification: (b) 60-120 mesh, (c) 100-200 mesh and (d) 230-300 mesh.

FIG. 31 illustrates the solidification process involving needles upon addition of silica gel (chromatographic grade): (a) 1 mL 50% aqueous NaOH+a needle, and after addition of silica gel for solidification: (b) 60-120 mesh, (c) 100-200 mesh and (d) 230-300 mesh.

FIG. 32 illustrates the solidification process involving solid swabs upon addition of silica gel (chromatographic grade): (a) 1 mL 50% aqueous NaOH+a swab, and after addition of silica gel for solidification: (b) 60-120 mesh, (c) 100-200 mesh and (d) 230-300 mesh.

FIG. 33 illustrates the solidification process involving tissue paper upon addition of alumina (chromatographic grade, basic, 60-325 mesh): (a) 1 mL 50% aqueous NaOH+a piece of tissue paper, and (b) after addition of alumina for solidification.

FIG. 34 illustrates the solidification process involving tissue paper upon addition of titania (mixture of anatase and rutile): (a) 1 mL 50% aqueous NaOH+a piece of tissue paper, and (b) after addition of titania for solidification.

FIG. 35 illustrates the photographs of the Petri dishes cultured with the samples taken (A,D) as control, (B,E) after addition of aqueous NaOH and (C,F) after solidification of an aqueous solution of NaOH and bacterial broths containing (A-C) E. coli, and (D-F) S. aureus confirming complete disinfection in quantitative experiments.

FIG. 36 illustrates the large scale solidification process involving a mixture of solid and liquid wastes upon addition of silica gel (chromatographic grade): (a) a mixture of solid and liquid wastes in 50% aqueous NaOH and (b) after addition of silica gel (60-120 mesh) for solidification.

FIG. 37 illustrates a prototype of an all-in-one sample collection-disinfection-solidification-disposal device for liquid samples, (a) consisting of three collection vials mounted one on top of the other such that (b) the top vial contains solid material A (silica is shown as an example), the middle one with collected sample and the bottom one prefilled with the requisite amount of solution B. Once collected sample is tested, the remaining sample could be initially disinfected by allowing (c) the sample to mix with solution B by breaking the junction between middle and bottom compartments followed by (d) solidification via the addition of material A by breaking the junction between the top and middle compartments.

FIG. 38 illustrates a prototype of an all-in-one sample collection-disinfection-solidification-disposal device for solid samples, (a) consisting of two collection vials mounted one on top of the other such that (b) the top vial contains solid material A (silica is shown as an example), and the bottom one prefilled with the requisite amount of solution B. The waste sample could be initially disinfected by (c) mixing the sample with solution B followed by (d) solidification via the addition of material A by breaking the junction between the two compartments.

FIG. 39 illustrates the design of the prototype of an all-in-one sample collection-disinfection-solidification-disposal device for liquid samples as shown in FIG. 37.

FIG. 40 illustrates the design of the prototype of an all-in-one sample collection-disinfection-solidification-disposal device for solid samples as shown in FIG. 38.

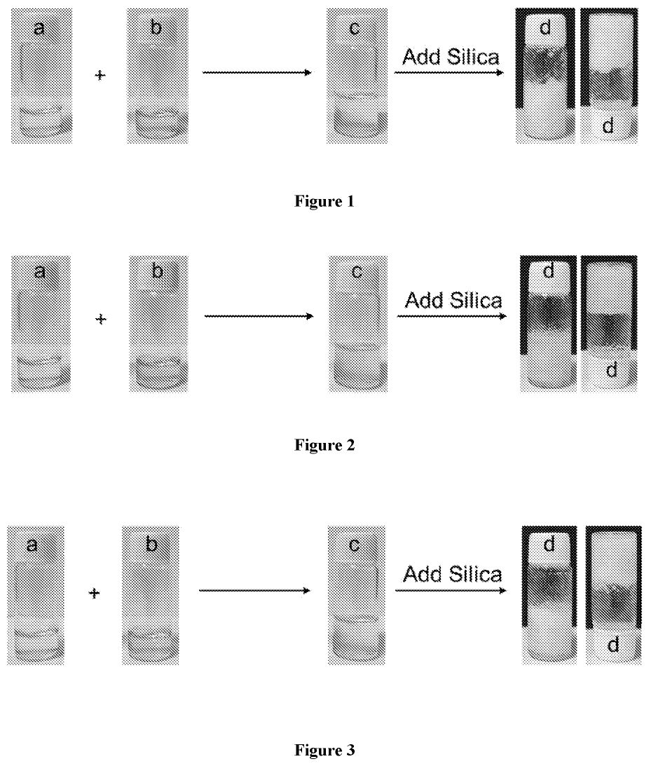

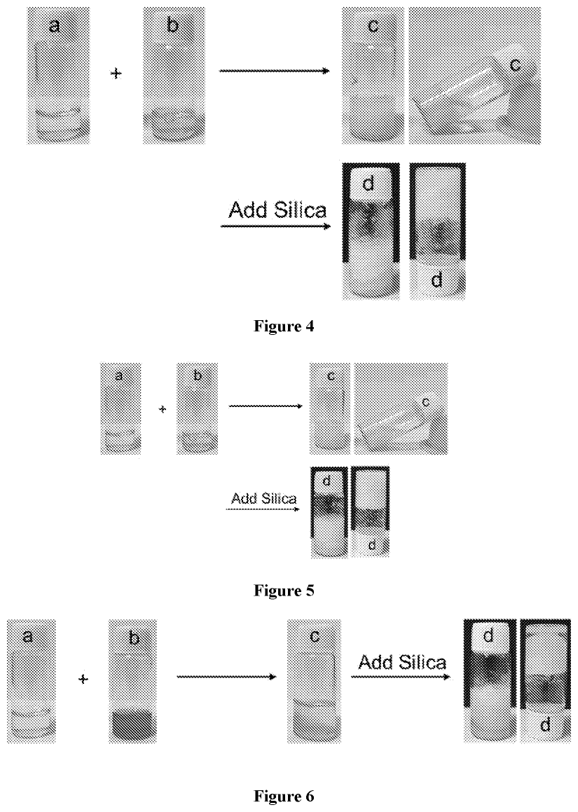

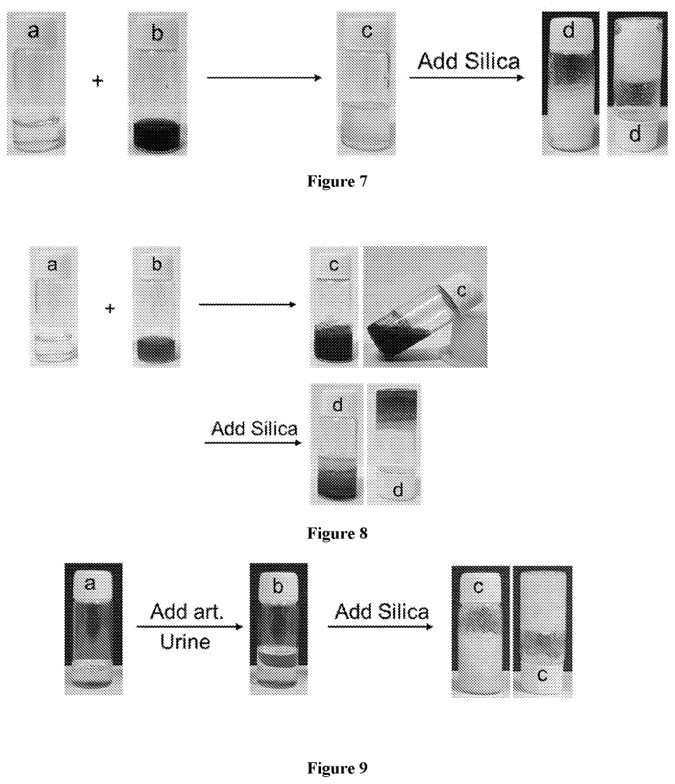

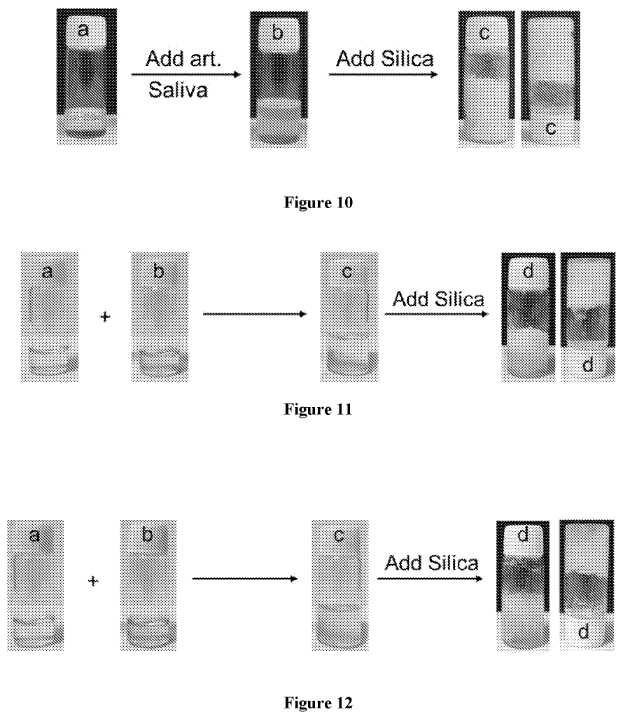

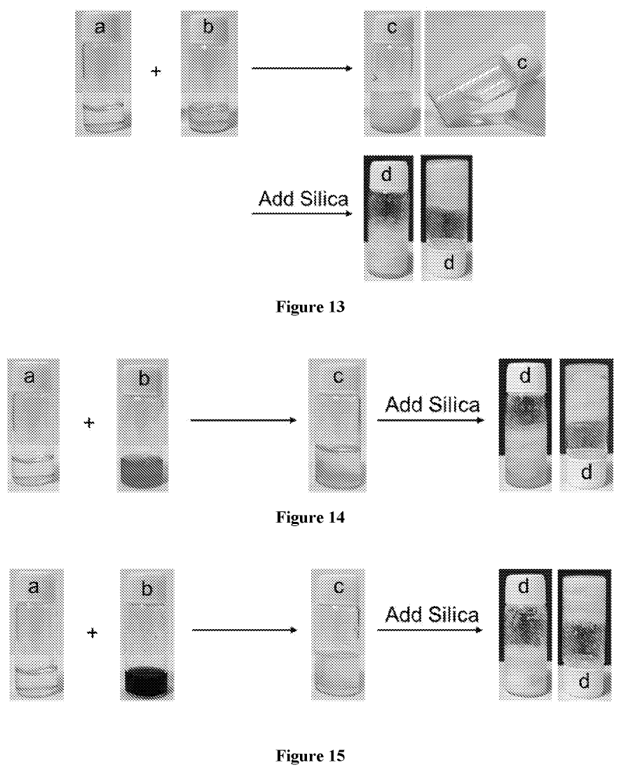

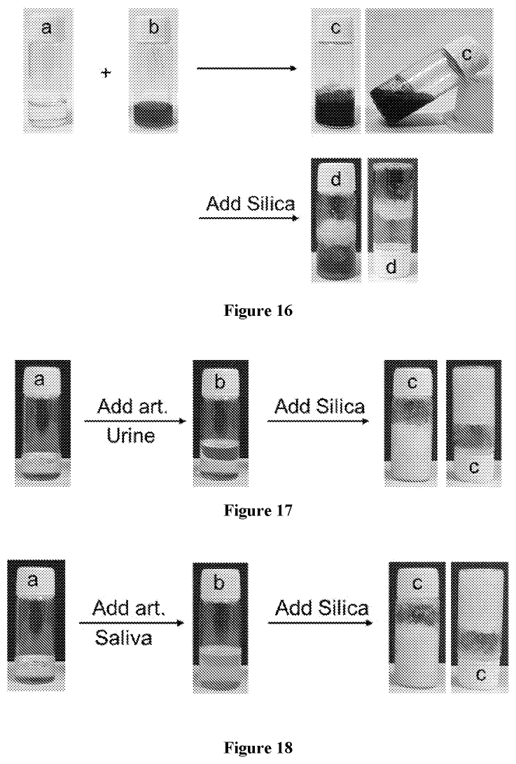

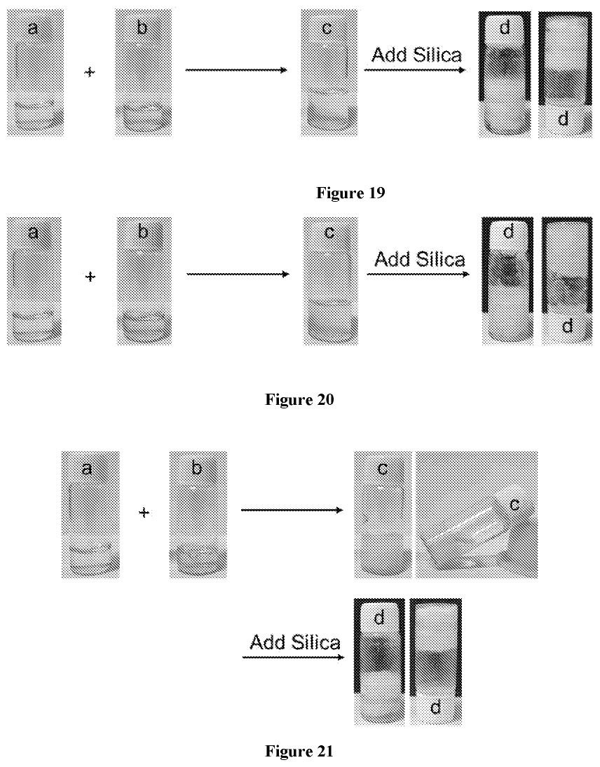

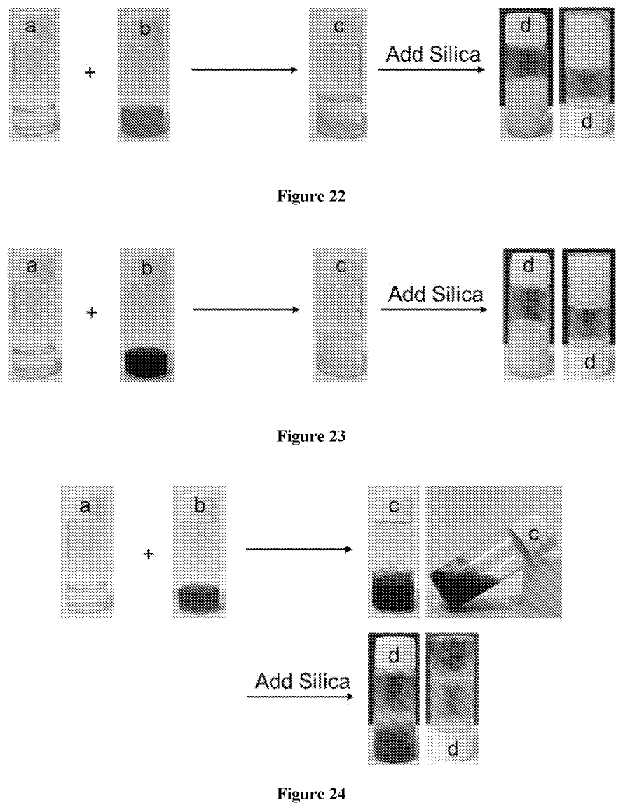

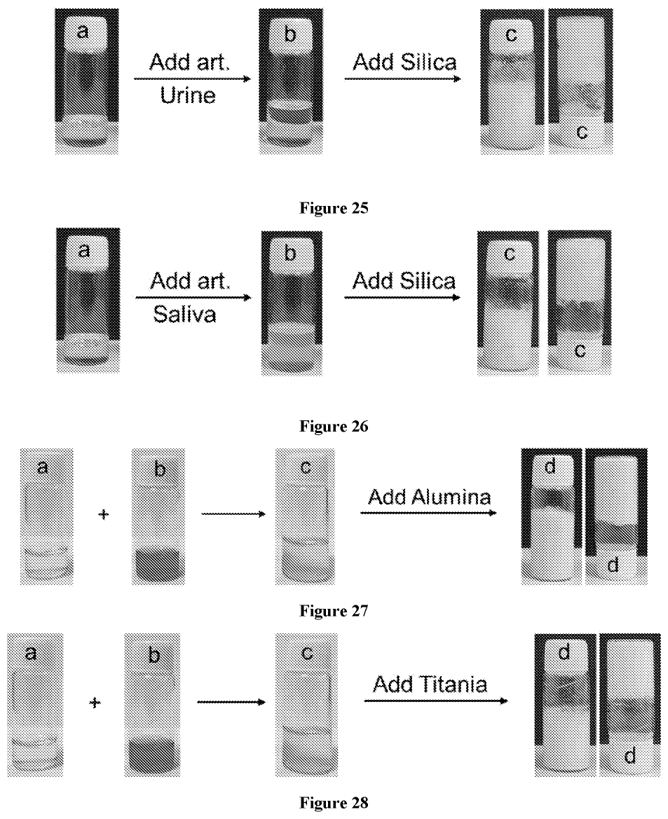

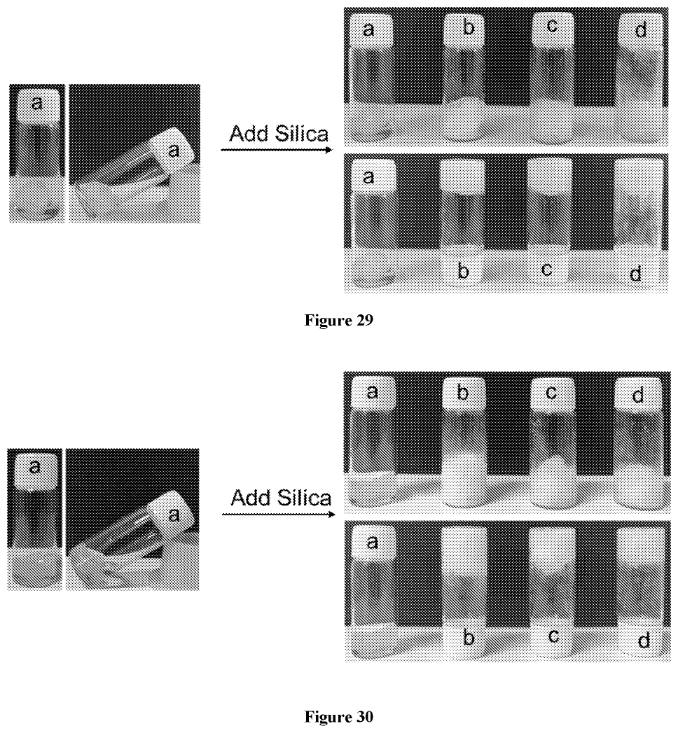

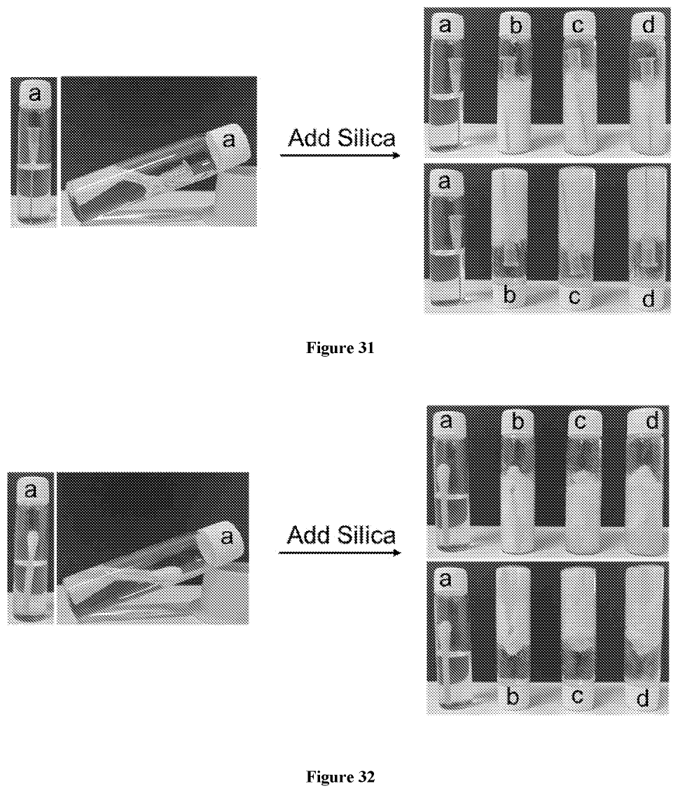

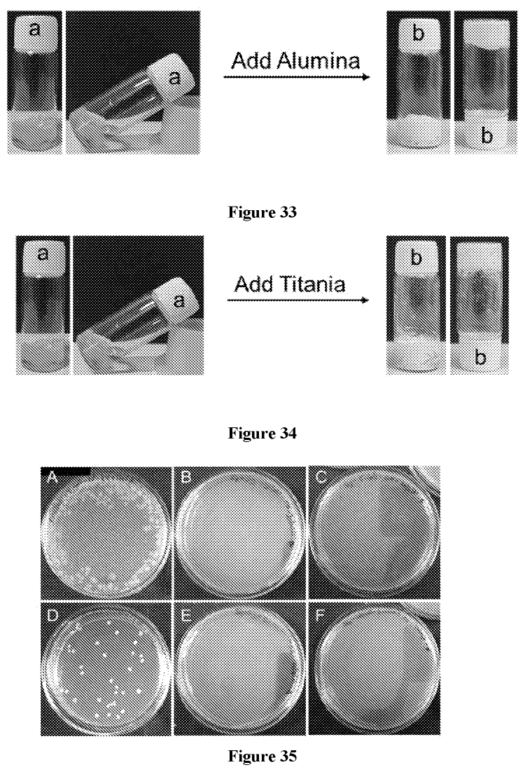

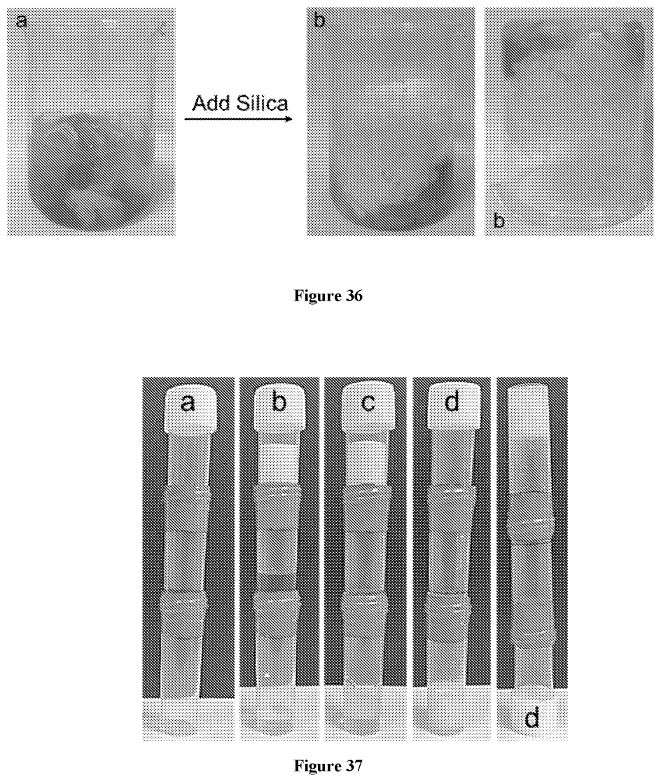

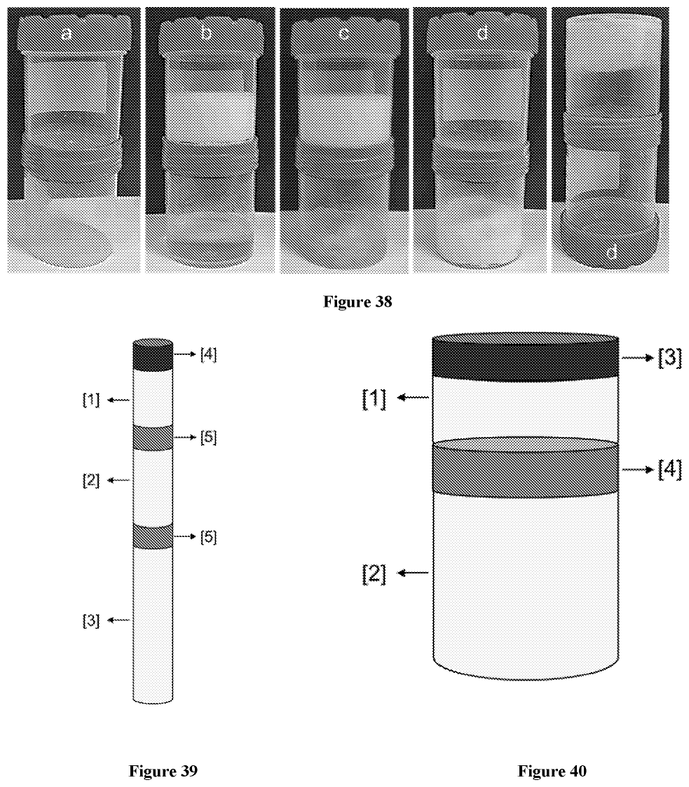

### DETAILED DESCRIPTION OF THE INVENTION

This section describes the present invention in preferred embodiments in detail. The attached illustrations/drawings are intended for the purpose of describing and understanding the preferred embodiments in detail and not to limit the invention or its scope or both thereto.

Upon extensive investigations, the inventors of the present invention previously found that adding a poly-amino acid as its aqueous solution to a stable nanomaterial sol in water leads to instantaneous flocculation and the said flocculation process could further be controlled to effect gelation or solidification under carefully controlled conditions. However, the use of sol and polyamino acids are not very effective for long term treatment and resting of biomedical waste and there is an urgent need to minimize the amount of water used, followed by reducing the number of chemical components. The present invention provides an improved process for the disinfection and solidification of pathogenic biomedical waste with reduced number of chemical components and minimal use of water.

The prime embodiment of the present subject matter provides an improved disinfection—solidification process for the preparation for disposal of solid and fluid wastes collected in a collection vessel at point of care, combined with the destruction, disinfection or deactivation of infectious agents including microorganisms inter alia bacteria, fungus etc., viruses and other toxins, whereby the disposal including treatment, handling and transportation are deemed easier, safer and cost-effective.

Solidification reduces the risk of spills and aerosolization, whereas complete pathogenic disinfection allows to dispose of the wastes thereof as non-regulated medical waste, which is less expensive than red-bagging. Segregation, transportation and incineration of such disinfected medical wastes are easier, safer and decrease medical waste disposal costs for a healthcare facility.

Another embodiment of the present invention comprises of the addition of oxides of transition metals inter alia titanium, aluminium, silicon or or zinc, with or without a binder, added to an aqueous solution basified to an alkaline pH using a base B containing the biomedical entity to be disinfected, such that the concentration of B is 0.1-90% w/v in water, more preferably >40% w/v in water and solid A is added at a minimum of 1% (w/v) and a maximum of 500% (w/v) of the total aqueous volume, resulting in instantaneous disinfection followed by instantaneous solidification.

The present invention intends to offer a self-disinfecting solidification process for the treatment and disposal of biomedical waste. The treatment process disclosed herein involves a solidifying agent inter alia silica powder with or without a binder, chromatography grade silica gel powder of 60-400 mesh size, alumina powder with or without a binder, chromatography grade alumina powder of 60-200 mesh size, titania powder with or without a binder, pigment grade titania in its rutile or anatase forms or a mixture of rutile and anatase forms, or zinc oxide powder with or without a binder, industrial grade zinc oxide in its powder form having particle size <500 μm, which when subjected to mixing with solid or fluid waste samples disinfected by adding to an alkaline solution of a base, at a defined volumetric and/or weighted composition leads to instantaneous solidification with up to 100% microbial disinfection.

In specific embodiments, the invention relates to providing a non-pourable environment for fluid medical wastes inter alia salt, sugar, saliva, urine, blood, hapital chemicals, etc. wherein risks related to spillage and occupational exposure are minimized, and further to the treatment of solid medical wastes inter alia cotton, tissue paper, swabs, needles, etc., wherein the risks related to accumulation of untreated and infected samples are minimized or a mixture of solid and liquid wastes added with >99.9% microbial disfection.

Another aspect of the present invention disclose the volumetric composition of an aqueous solution of a pH regulating base or alkali for complete disinfection of fluid or solid medical waste followed by the addition of an oxide based solid powder, as a single or plurality of the said powders, for instanteneous solidification of solid or fluid samples containing proteins, microbial cultures, salt or metal ions in high concentrations.

Another aspect of the present invention is directed to creating all-in-one sample collection—disinfection—solidification devices of requisite dimensions capable of collecting the solid or liquid sample, flocculating/gelating/solidifying the samples as and when required and disinfecting the same for preparation for its disposal. and immobilizing them as and when required with prior pathogenic disinfection for preparation for its disposal.

In an embodiment, the present invention provides a process for disinfection followed by solidification by disinfection-solidification and disposal system, said process comprising the steps of adding disinfection composition comprising solid powders of a solidifying agent A and basifying agent B, wherein oxide based powders inter alia oxides of silicon, titanium, zinc or aluminium are added as solid powders from solidifying agent A, to an aqueous solution basified to an alkaline pH in the range of 9 to 14 using the basifying agent B containing the biomedical waste to be disinfected, wherein solid powders of the solidifying agent A is added at a minimum of 1% (w/v) and a maximum of 500% (w/v) of the total aqueous volume and the concentration of the basifying agent B is 1-90% w/v in water, more preferably >40% w/v in water.

In yet another embodiment, the solid powders of the solidifying agent A is silica powder with or without a binder, chromatography grade silica gel powder of 60-400 mesh size, alumina powder with or without a binder, chromatography grade alumina powder of 60-400 mesh size, titania powder with or without a binder, pigment grade titania in its rutile or anatase forms or a mixture of rutile and anatase forms, or zinc oxide powder with or without a binder, industrial grade zinc oxide in its powder form having particle size <500 μm.

Further, said basifying agent B is selected from hydroxides of alkali or alkaline earth metals selected from the group comprising of sodium or potassium hydroxide, basic salts of metals and organic cations, leading to a final pH in the range 9-14 in its aqueous solution.

In yet another embodiment, the present invention relates to a process for disinfection-solidification, comprising the steps of:
(a) preparation of an aqueous solution of basifying agent B in water;
(b) addition of the biomedical waste to be disinfected to the said aqueous solution as prepared in step (a);
(c) homogeneous mixing of the mixture obtained in step (b) and/or resting for 10-30 min; and
(d) addition of solid powders of a solidifying agent A followed by mixing and/or resting, wherein the obtained mixture is characterized as solidified or gelled depending on the amounts of the solidifying agent A, basifying agent B and the biomedical waste.

Further, the amount of the biomedical waste added is less than 1:1000 (v/v) of basifying agent solution B for liquid waste and any immersible amount of solid waste or a mixture thereof. The solid powders of a solidifying agent A is added at a minimum of 1% (w/v) and a maximum of 500% (w/v) of the total aqueous volume in the mixture.

In yet another embodiment of the present invention, the present invention relates to a process for disinfection-solidification, wherein the biomedical waste used in step (b) is selected from the group consisting of salt, sugar, metal salts and complexes, aqueous waste, hospital chemicals such as iodine, saliva, urine, blood or any solid sample, inter alia cotton, tissue paper, needle, syringes or swabs alone or in combination thereof, whereby disinfection is effected by the high pH of basifying agent solution B.

Further, the exothermic reaction between the solid powders of the solidifying agent A and the alkaline waste mixture provides a secondary thermal mechanism for pathogenic disinfection, said exothermicity is in the range 50-120° C.

In yet another embodiment, the present invention provides a disinfection-solidification and disposal system filled with the disinfected composition, the device comprising of:
(a) an upper container or compartment system [1, FIG. 39];
(b) a middle container or compartment system [2, FIG. 39];
(c) a bottom container or compartment system [3, FIG. 39];
(d) a screw cap [4, FIG. 39] connected to the upper container or compartment system;
(e) two breakable screw-caps [5, FIG. 39], one connected between the upper and the middle container or compartment systems and another connected between the middle and the bottom container or compartment systems.

Further, the upper container or compartment system is filled with solid powder of the solidifying agent A in the disinfection-solidification and disposal system. The middle container or compartment system is filled with the biomedical waste. The bottom container or compartment system is filled with the aqueous solution of basifying agent B. Further, the biomedical waste is solid or liquid waste or their mixture.

In yet another embodiment, the present invention provides a disinfection composition comprising:
a) solid powders of a solidifying agent A, wherein oxide based powders inter alia oxides of silicon, titanium, zinc or aluminium are added as solid powders from solidifying agent A; solid powders of the solidifying agent A is added at a minimum of 1% (w/v) and a maximum of 500% (w/v) of the total aqueous volume; and
b) basifying agent B, wherein the concentration of the basifying agent B is 1-90% w/v in water, more preferably >40% w/v in water;
wherein solid powders of a solidifying agent A is added to an aqueous solution basified to an alkaline pH in the range of 9 to 14 using the basifying agent B containing the biomedical waste to be disinfected.

### EXAMPLES

Following examples are given by way of illustration and therefore should not be construed to limit the scope of the invention.

### Example 1. Solidification of Aqueous Waste Using Silica Gel Powder (60-120, 100-200 or 230-400 Mesh)

50% NaOH solution was made in water. The aqueous waste (1:1) was added to the above solution and mixed well. Solid silica powder was added to effect instantaneous solidification.

### Example 2. Solidification of Concentrated Salt Solution Using Silica Gel Powder (60-120, 100-200 or 230-400 Mesh)

50% NaOH solution was made in water. A saturated aqueous solution of sodium chloride (1:1) was added to the above solution and mixed well. Solid silica powder was added to effect instantaneous solidification.

### Example 3. Solidification of Concentrated Sugar Solution Using Silica Gel Powder (60-120, 100-200 or 230-400 Mesh)

50% NaOH solution was made in water. A saturated aqueous solution of sucrose (1:1) was added to the above solution and mixed well. Solid silica powder was added to effect instantaneous solidification.

### Example 4. Solidification of a Mixture of Concentrated Salt and Sugar Solutions Using Silica Gel Powder (60-120, 100-200 or 230-400 Mesh)

50% NaOH solution was made in water. A mixture of saturated aqueous solutions of sodium chloride and sucrose (1:1) was added to the above solution and mixed well. Solid silica powder was added to effect instantaneous solidification.

### Example 5. Solidification of Aqueous Waste Containing Proteins Using Silica Gel Powder (60-120, 100-200 or 230-400 Mesh)

50% NaOH solution was made in water. A 6% aqueous solution of BSA (1:1) was added to the above solution and mixed well. Solid silica powder was added to effect instantaneous solidification. Full Form of BSA is Bovine Serum Albumin.

### Example 6. Solidification of Concentrated Salt Solution Containing Proteins Using Silica Gel Powder (60-120, 100-200 or 230-400 Mesh)

50% NaOH solution was made in water. A saturated aqueous solution of sodium chloride containing 6% BSA (1:1) was added to the above solution and mixed well. Solid silica powder was added to effect instantaneous solidification.

### Example 7. Solidification of Aqueous Solution Containing Metal Ions and Harsh Oxidising Agent Using Silica Gel Powder (60-120, 100-200 or 230-400 Mesh)

50% NaOH solution was made in water. A saturated aqueous solution of potassium dichromate (1:1) was added to the above solution and mixed well. Solid silica powder was added to effect instantaneous solidification.

### Example 8. Solidification of Aqueous Waste Containing Hospital Chemicals Using Silica Gel Powder (60-120, 100-200 or 230-400 Mesh)

50% NaOH solution was made in water. A concentrated aqueous solution of iodine (1:1) was added to the above solution and mixed well. Solid silica powder was added to effect instantaneous solidification.

### Example 9. Solidification of Aqueous Wastes Using Alumina Powder (60-400 Mesh)

50% NaOH solution was made in water. The aqueous waste as mentioned in examples 1-8 above (1:1) was added to the above solution and mixed well. Solid alumina powder was added to effect instantaneous solidification.

### Example 10. Solidification of Aqueous Wastes Using Titania Powder (Mixture of Anatase and Rutile)

50% NaOH solution was made in water. The aqueous waste as mentioned in examples 1-8 above (1:1) was added to the above solution and mixed well. Solid titania powder was added to effect instantaneous solidification.

### Example 11. Solidification of Aqueous Wastes Using Zinc Oxide Powder (Particle Size <500 μm)

50% NaOH solution was made in water. The aqueous waste as mentioned in examples 1-8 above (1:1) was added to the above solution and mixed well. Solid zinc oxide powder was added to effect instantaneous solidification.

### Example 12. Preparation of Artificial Saliva

Artificial saliva was prepared according to the following two procedures: (i) Mixing 1.5 mM Ca(NO₃)₂, 0.90 mM KH₂PO₄, 130 mM KCl and 60 mM Tris buffer at pH 7.4 (Reference may be made to:Kirkham, J.;et al., Self-assembling peptide scaffolds promote enamel remineralization,J.Dental Res. 2007,86, 426-430). (ii) Mixing sodium chloride (0.06 g), potassium chloride (0.072 g), calcium chloride dihydrate (0.022 g), potassium dihydrogen phosphate (0.068 g), disodium hydrogen phosphate dodecahydrate (0.086 g), potassium thiocyanate (0.006 g), sodium hydrogen carbonate (0.15 g), and citric acid (0.003 g) in 100 mL distilled water at pH 6.5 (Reference may be made to:Duffó, G. S.; et al., Development of an artificial saliva solution for studying the corrosion behavior of dental alloys. Corrosion 2004, 60,594-602).

### Example 13. Solidification of Artificial Saliva Using Silica Gel Powder (60-120, 100-200 or 230-400 Mesh)

50% NaOH solution was made in water. Artificial saliva (1:1) was added to the above solution and mixed well. Solid silica gel powder was added to effect instantaneous solidification.

### Example 14. Solidification of Artificial Saliva Using Alumina Powder (60-400 Mesh)

50% NaOH solution was made in water. Artificial saliva (1:1) was added to the above solution and mixed well. Solid alumina powder was added to effect instantaneous solidification.

### Example 15. Solidification of Artificial Saliva Using Titania Powder (Mixture of Anatase and Rutile)

50% NaOH solution was made in water. Artificial saliva (1:1) was added to the above solution and mixed well. Solid titania powder was added to effect instantaneous solidification.

### Example 16. Solidification of Artificial Saliva Using Zinc Oxide Powder (Particle Size <500 m)

50% NaOH solution was made in water. Artificial saliva (1:1) was added to the above solution and mixed well. Solid zinc oxide powder was added to effect instantaneous solidification.

### Example 17. Preparation of Artificial Urine

To 75 mL of distilled water in a container, urea (1.82 g) was added and shaken well to dissolve. Sodium chloride (0.75 g), potassium chloride (0.45 g) and sodium phosphate (0.48 g) were further added to the above mixture and mixed well until dissolved. The pH was adjusted to be between 5 and 7. Creatinine (200 mg) and albumin powder (5 mg)were added and mixed gently. The artificial urine thus obtained was further spiked with a few mg of glucose before each experiment.

### Example 18. Solidification of Artificial Urine Using Silica Gel Powder (60-120, 100-200 or 230-400 Mesh)

50% NaOH solution was made in water. Artificial urine (1:1) was added to the above solution and mixed well. Solid silica gel powder was added to effect instantaneous solidification.

### Example 19. Solidification of Artificial Urine Using Alumina Powder (60-400 Mesh)

50% NaOH solution was made in water. Artificial urine (1:1) was added to the above solution and mixed well. Solid alumina powder was added to effect instantaneous solidification.

### Example 20. Solidification of Artificial Urine Using Titania Powder (Mixture of Anatase and Rutile)

50% NaOH solution was made in water. Artificial urine (1:1) was added to the above solution and mixed well. Solid titania powder was added to effect instantaneous solidification.

### Example 21. Solidification of Artificial Urine Using Zinc Oxide Powder (Particle Size <500 μm)

50% NaOH solution was made in water. Artificial urine (1:1) was added to the above solution and mixed well. Solid zinc oxide powder was added to effect instantaneous solidification.

### Example 22. Preparation of Artificial Blood

A 6% solution of BSA was prepared in distilled water. A small amount of an iron(II) complex was added to mimic heme and impart color. Full form of BSA is Bovine Serum Albumin.

### Example 23. Solidification of Artificial Blood Using Silica Gel Powder (60-120, 100-200 or 230-400 Mesh)

50% NaOH solution was made in water. Artificial blood (1:1) was added to the above solution and mixed well. Solid silica gel powder was added to effect instantaneous solidification.

### Example 24. Solidification of Artificial Blood Using Alumina Powder (60-400 Mesh)

50% NaOH solution was made in water. Artificial blood (1:1) was added to the above solution and mixed well. Solid alumina powder was added to effect instantaneous solidification.

### Example 25. Solidification of Artificial Blood Using Titania Powder (Mixture of Anatase and Rutile)

50% NaOH solution was made in water. Artificial blood (1:1) was added to the above solution and mixed well. Solid titania powder was added to effect instantaneous solidification.

### Example 26. Solidification of Artificial Blood Using Zinc Oxide Powder (Particle Size <500 m)

50% NaOH solution was made in water. Artificial blood (1:1) was added to the above solution and mixed well. Solid zinc oxide powder was added to effect instantaneous solidification.

### Example 27. Immobilization of a Solid Swab in Silica Gel (60-400 Mesh), Alumina (60-400 Mesh), Titamia (Mixture of Anatase and Rutile) or Zinc Oxide (Particle Size <500 μm) Powders

50% NaOH solution was made in water in an 8 mL glass vial and a piece of swab (4 cm) was immersed. It was mixed well and solid powder of silica gel (60-400 mesh), alumina (60-400 mesh), titamia (mixture of anatase and rutile) or zinc oxide (particle size <500 m) was added, resulting in instantaneous solidification.

### Example 28. Immobilization of a Syringe Needle in Silica Gel (60-400 Mesh), Alumina (60-400 Mesh), Titamia (Mixture of Anatase and Rutile) or Zinc Oxide (Particle Size <500 μm) Powders

50% NaOH solution was made in water in an 8 mL glass vial and a needle (4-6 cm) was immersed. It was mixed well and solid powder of silica gel (60-400 mesh), alumina (60-400 mesh), titamia (mixture of anatase and rutile) or zinc oxide (particle size <500 m) was added, resulting in instantaneous solidification.

### Example 29. Immobilization of Cotton Waste in Silica Gel (60-400 Mesh), Alumina (60-400 Mesh), Titamia (Mixture of Anatase and Rutile) or Zinc Oxide (Particle Size <500 μm) Powders

50% NaOH solution was made in water in a glass vial and a piece of waste cotton was immersed. It was mixed well and solid powder of silica gel (60-400 mesh), alumina (60-400 mesh), titamia (mixture of anatase and rutile) or zinc oxide (particle size <500 m) was added, resulting in instantaneous solidification.

### Example 30. Immobilization of Tissue Paper in Silica Gel (60-400 Mesh), Alumina (60-400 Mesh), Titamia (Mixture of Anatase and Rutile) or Zinc Oxide (Particle Size <500 μm) Powders

50% NaOH solution was made in water in a glass vial and a piece of tissue paper was immersed. It was mixed well and solid powder of silica gel (60-400 mesh), alumina (60-400 mesh), titamia (mixture of anatase and rutile) or zinc oxide (particle size <500 m) was added, resulting in instantaneous solidification.

### Example 31. Immobilization of Large Scale Mixed Waste in Silica Gel (60-400 Mesh), Alumina (60-400 Mesh), Titamia (Mixture of Anatase and Rutile) or Zinc Oxide (Particle Size <500 μm) Powders

50% NaOH solution was made in water in a glass beaker and a mixture of different wastes (solid and liquid—syringe, needle, swab, cotton, tissue, artificial urine, blood and saliva, iodine, potassium dichromate, salt, sugar, etc.) was added. It was mixed well and solid powder of silica gel (60-400 mesh) was added, resulting in instantaneous solidification.

### Example 32. Antimicrobial Studies

Cultures of Escherichia coli and Staphylococcus aureus were prepared in Luria Bertiani (LB) medium and taken for test at 18 h. old stage where the colony forming units (cfus) are approximately 1-3×10⁶ per millilitre for E. coli or S. aureus. (previously standardized based on optical densities at 600 nm). 1 mL of 50% aqueous solution of base B was added to 1 mL of the bacterial broath (spiking solution) and mixed by swirling the bottle. Samples were taken for analysis after regular intervals of time. Solid powder of silica gel (60-120 mesh) was added to effect instanteneous solidification. Samples were further taken for analysis after regular intervals of time. All samples were taken as diluted 10× in sterile saline and 100 μL of the diluted solution was plated onto LB agar plates andincubated over night at 37° C. Parallely, the original bacterial suspension was diluted serially in sterile saline and 100 μL of the appropriate dilutions were plated on LB agar plates and incubated as for the test sample that served as controls. Colonies were counted the next day and based on applied dilution, the number of CFUs/mL of the original bacterial suspension added to the sol and the CFUs in the gelled disinfectant were calculated. Efficiency was calculated as follows:[(Number of CFUs in Bacterial suspension−Number of CFUs in the gelled disinfectant)/Number of CFUs in Bacterial suspension]×100 and expressed in %.

### Example 33. Prototype for All-In-One Sample Collection-Disinfection-Disposal Devices for Fluid Samples

An all-in-one sample collection-disinfection-disposal device for fluid samples was prototyped as follows: Three plastic collection vials were mounted one on top of the other such that the top vial contained solid powder of silica gel (60-400 mesh), alumina (60-400 mesh), titamia (mixture of anatase and rutile) or zinc oxide (particle size <500 m), the middle one for sample collection and the bottom one prefilled with the requisite amount of 50% aqueous solution of sodium hydroxide. The design allows the top compartment to be unscrewed and the samples could be collected in the middle compartment. Once collected sample is tested, the remaining sample could be disinfected and solidified by initially allowing the sample to mix with the alkaline solution in the bottom container by breaking the junction between the middle and bottom compartments followed by the addition of the corresponding solid powder from the top compartment by breaking the junction between the top and middle compartments. The mixing of the three fluid mixtures allow for complete pathogenic disinfection as evidenced in Example 32.

### Example 34. Prototype for All-In-One Sample Collection-Disinfection-Disposal Devices for Solid Samples

An all-in-one sample collection-disinfection-disposal device for solid samples was prototyped as follows: A plastic collection container for solid samples (Eg: cotton waste) was mounted on its top with another plastic vial such that the top vial contained silica gel (60-400 mesh), alumina (60-400 mesh), titamia (mixture of anatase and rutile) or zinc oxide (particle size <500 m), and the bottom one was prefilled with the requisite amount of 50% aqueous solution of sodium hydroxide. The design allows the top compartment to be unscrewed and the solid samples could be collected in the bottom compartment. Once ample number of solid samples are collected in the bottom container, it could be disinfected and solidified by allowing the alkaline sample to mix with the corresponding solid powders by breaking the junction between the two compartments. The mixing of the solutions and gelation allow for complete pathogenic disinfection as evidenced in Example 32.

### Advantages of the Invention

Inherent antimicrobial activity
Minimized amount of water required
Instantaneous disinfection and solidification upon mixing
>99.9% microbial disinfection within 1 minute
Reduces risks of spillage and occupational exposure
Allows to dispose the waste as non-regulated medical waste
Applicable to both fluid as well as solid medical waste decontamination
Safer, easier and cost-effective
Adaptability to manage any amount of fluidic waste
No interference from proteins, metal ions, salt or other impurities

1. A process for disinfection followed by solidification by disinfection-solidification and disposal system, said process comprising the steps of adding disinfection composition comprising solid powders of a solidifying agent A and basifying agent B,
wherein oxide based powders inter alia oxides of silicon, titanium, zinc or aluminium are added as solid powders from solidifying agent A, to an aqueous solution basified to an alkaline pH in the range of 9 to 14 using the basifying agent B containing the biomedical waste to be disinfected, wherein solid powders of the solidifying agent A is added at a minimum of 1% (w/v) and a maximum of 500% (w/v) of the total aqueous volume and the concentration of the basifying agent B is 1-90% w/v in water, more preferably >40% w/v in water.

2. The process as claimed in claim 1, wherein the solid powders of the solidifying agent A is silica powder with or without a binder, chromatography grade silica gel powder of 60-400 mesh size, alumina powder with or without a binder, chromatography grade alumina powder of 60-400 mesh size, titania powder with or without a binder, pigment grade titania in its rutile or anatase forms or a mixture of rutile and anatase forms, or zinc oxide powder with or without a binder, industrial grade zinc oxide in its powder form having particle size <500 μm.

3. The process as claimed in claim 1, where the said basifying agent B is selected from hydroxides of alkali or alkaline earth metals selected from the group comprising of sodium or potassium hydroxide, basic salts of metals and organic cations, leading to a final pH in the range 9-14 in its aqueous solution.

4. The process for disinfection-solidification as claimed in claim 1, comprising the steps of:
(a) preparation of an aqueous solution of basifying agent B in water;
(b) addition of the biomedical waste to be disinfected to the said aqueous solution as prepared in step (a);
(c) homogeneous mixing of the mixture obtained in step (b) and/or resting for 10-30 min; and
(d) addition of solid powders of a solidifying agent A followed by mixing and/or resting, wherein the obtained mixture is characterized as solidified or gelled depending on the amounts of the solidifying agent A, basifying agent B and the biomedical waste.

5. The process for disinfection-solidification as claimed in claim 4, wherein in the amount of the biomedical waste added is less than 1:1000 (v/v) of basifying agent solution B for liquid waste and any immersible amount of solid waste or a mixture thereof.

6. The process for disinfection-solidification as claimed in claim 4, wherein solid powders of a solidifying agent A is added at a minimum of 1% (w/v) and a maximum of 500% (w/v) of the total aqueous volume in the mixture.

7. The process for disinfection-solidification as claimed in claim 4, wherein the biomedical waste used in step (b) is selected from the group consisting of salt, sugar, metal salts and complexes, aqueous waste, hospital chemicals such as iodine, saliva, urine, blood or any solid sample, inter alia cotton, tissue paper, needle, syringes or swabs alone or in combination thereof, whereby disinfection is effected by the high pH of basifying agent solution B.

8. The process for disinfection-solidification as claimed in claim 4, wherein the exothermic reaction between the solid powders of the solidifying agent A and the alkaline waste mixture provides a secondary thermal mechanism for pathogenic disinfection, said exothermicity is in the range 50-120° C.

9. A disinfection-solidification and disposal system filled with the disinfected composition as claimed in claim 1, the device comprising of:
(a) an upper container or compartment system;
(b) a middle container or compartment system;
(c) a bottom container or compartment system;
(d) a screw cap connected to the upper container or compartment system;
(e) two breakable screw-caps, one connected between the upper and the middle container or compartment systems and another connected between the middle and the bottom container or compartment systems.

10. The disinfection-solidification and disposal system as claimed in claim 9, wherein the upper container or compartment system is filled with solid powder of the solidifying agent A.

11. The disinfection-solidification disposal system as claimed in claim 9, wherein the middle container or compartment system is filled with the biomedical waste.

12. The disinfection-solidification and disposal system as claimed in claim 9, wherein the bottom container or compartment system is filled with the aqueous solution of basifying agent B.

13. The disinfection-solidification and disposal system as claimed in claim 11, wherein the biomedical waste is solid or liquid waste or their mixture.

14. A disinfection composition comprising:
a) solid powders of a solidifying agent A, wherein oxide based powders inter alia oxides of silicon, titanium, zinc or aluminium are added as solid powders from solidifying agent A; solid powders of the solidifying agent A is added at a minimum of 1% (w/v) and a maximum of 500% (w/v) of the total aqueous volume; and
b) basifying agent B, wherein the concentration of the basifying agent B is 1-90% w/v in water, more preferably >40% w/v in water;
wherein solid powders of a solidifying agent A is added to an aqueous solution basified to an alkaline pH in the range of 9 to 14 using the basifying agent B containing the biomedical waste to be disinfected.
